SEQUENCE LISTING (39 sequences)

SEQ ID NO: 1 - protein, 49 aa, Artificial sequence
  misc_feature (1)..(49): Consensus sequence for polypeptides of the invention
XXXXYXXYXXXXXXXXPCXXXGXSYYNCXXXXXANPYXXXCXXIXXCXX

SEQ ID NO: 2 - DNA, 543 bp, Nicotiana tabacum
  CDS (131)..(475)
ggcaccagggaagtttcaacagcgaaaacatagtaaaaagataccaaaaaccagcggtccaaagcataaacaggcagaaatagcaaaagggtgttttaatttctcatcattttttcagtacaaggtaaaaatgggagttccttcaggtttgattctttgtgttctgatcggagcttttttcatttcaatggcggcggccggagatagtggggcctacgattgggtgatgccggcgagatctggtgggggatgcaaagggagtatcggagagtgcattgctgaagaagaggagtttgagctggacagtgagtcaaacaggcgcattttagccaccaaaaagtacatcagctatggtgcactgcagaagaacagtgtaccttgttctcgccgtggagcttcgtattacaactgcaaacctggtgctcaggctaatccttactctcgtggatgcagtgctatcactcgttgcaggagttaagttctgaatttctcttcttcttccacagaatccaaaaatattgaatttttcatggaaaattgaat

SEQ ID NO: 3 - protein, 115 aa, Nicotiana tabacum
MGVPSGLILCVLIGAFFISMAAAGDSGAYDWVMPARSGGGCKGSIGECIAEEEEFELDSESNRRILATKKYISYGALQKNSVPCSRRGASYYNCKPGAQANPYSRGCSAITRCRS

SEQ ID NO: 4 - protein, 49 aa, Lycopersicon esculentum
ATKKYISYGALQKNSVPCSRRGASYYNCKPGAQANPYTRGCSAITRCRS

SEQ ID NO: 5 - protein, 17 aa, Nicotiana tabacum
ATKKYISYGALQKNSVP

SEQ ID NO: 6 - protein, 49 aa, Nicotiana tabacum
ATKKYISYGALQKNSVPCSRRGASYYNCKPGAQANPYSRGCSAITRCRS

SEQ ID NO: 7 - protein, 49 aa, Pisum sativum
ATTKYISYGALQRNTVPCSRRGASYYNCRPGAQANPYSRGCSAITRCRS

SEQ ID NO: 8 - protein, 49 aa, Medicago truncatula
ATTKYISYGALQRNTVPCSRRGASYYNCRPGAQANPYSRGCSAITRCRG

SEQ ID NO: 9 - protein, 49 aa, Gossypium hirsutum
QTTRYISYGALQRNTVPCSRRGASYYNCQPGAQANPYNRGCSRITRCRG

SEQ ID NO: 10 - protein, 49 aa, Populus tremula x, populus tremuloides
ATSSYVSYGALQKNNVPCSRRGASYYNCKNGAQANPYSRGCSRITRCRG

SEQ ID NO: 11 - protein, 49 aa, Arabidopsis thaliana
ATTKYISYQSLKRNSVPCSRRGASYYNCQNGAQANPYSRGCSKIARCRS

SEQ ID NO: 12 - protein, 49 aa, Mesembryanthemum cystallinum
ATNSYISYGALNKNRVPCSRRGASYYNCRPGAQANPYSRGCSRITRCRP

SEQ ID NO: 13 - protein, 49 aa, Glycine max
AGRSYISYGALRRNTVPCSRRGASYYNCRPGAQANPYSRGCSAITRCRR

SEQ ID NO: 14 - protein, 49 aa, Oryza sativa
GGSGYIGYGALRRDSVPCSQRGASYYNCQPGAEANPYSRGCSAITQCRG

SEQ ID NO: 15 - protein, 49 aa, Triticum aestivum
DGSGYIGYGALRRDNVPCSQRGASYYNCQPGAEANPYSRGCSAITQCRG

SEQ ID NO: 16 - protein, 49 aa, Zea mays
YGGGYISYGALRRDNVPCSRRGASYYNCRPGGQANPYHRGCSRITRCRG

SEQ ID NO: 17 - protein, 49 aa, Sorghum bicolor
YGNGYISYGALRRDNVPCSRRGASYYNCRPGGQANPYHRGCSRITRCRG

SEQ ID NO: 18 - protein, 49 aa, Hordeum vulgare
QGRGYISYGALRRGTVPCNRRGASYYNCRPGAQANPYHRGCSRITRCRG

SEQ ID NO: 19 - protein, 49 aa, Cryptomeria japonica
ATTQYISYGALRADSVPCSKSGTSYYNCGSSGQANPYSKSCTQITRCAR

SEQ ID NO: 20 - protein, 49 aa, Pinus taeda
AGRTYISYKSLAADSVPCSKRGTSYYNCRSTSQANPYQRSCTQITRCAR

SEQ ID NO: 21 - DNA, 342 bp, Lycopersicon esculentum
atgggagtttcttcgtatttgattgtttgtgttcttgttggagcttttttcatttccatggctgccgccggcgacagtggtagctacgattggatggtgccggcgagatccggtgaatgcaaggggagtattgcagagtgcatggctgaagaagatgagtttgcccttgacagtgagtcaaacaggcgtattttagcaaccaaaaagtacatcagctatggtgcactccagaagaacagtgtgccgtgttctcgccgtggagcttcctactacaactgcaaacctggagctcaagcaaatccctacactcgtggatgcagtgctattactcgttgcaggagc

SEQ ID NO: 22 - DNA, 372 bp, Pisum sativum
atggcaccaccaagcttctattcgcacctcctcctcataatttgcgccaccgtcttgttaacactaacgatttcaccaccgacagtagatgccggaggattcaacttcggaatggaatggattcaccaaaccaaaacaacctgcgaaggttccatcgccgattgcatgttgcaacaaggtgaagaagagtttcagtttgataacgagatcaacaggcgtattttagcaacaacaaagtacatcagctacggtgcgcttcagaggaacactgtgccatgctctcgccgtggcgcctcgtactacaattgccgtcccggtgctcaggccaacccttacagccgtggatgcagcgctatcacgaggtgccggagt

SEQ ID NO: 23 - DNA, 381 bp, Medicago truncatula
atggcatcgaatttctactctcaactcttcctcgtgatttgtgccacccttttgatgacaacgatgatgagttcttcaccaacagtagatgcagctggaggattcgaacttggaggaatggaatggattcatcaaacaaaaacagccacatgcgaaggctccatcgctgattgcatgttacaacaaggtgaagaagagtttcagtttgataatgaaatcaacaggcgtatcttagcaaccacaaagtacatcagctatggtgcgcttcagaggaacactgtcccatgttctcgccgtggtgcttcttactacaattgcagacccggtgctcaggctaacccttacagtcgtggatgcagtgctattacgaggtgcaggggt

SEQ ID NO: 24 - DNA, 372 bp, Gossypium hirsutum
atggcagtgctcaattcttgtaagcttgtttggatctgcgccgtgatcgtggcggcggcgttgatggtggcagttgatgcgagcggtgaccaccactaccacaaccagcaaatcctgggttggattccgacccccaccagatcttcttgcaatggttccataggagaatgcttaggaggggaagaagagtttgagctggactcggagatcagccgccgcgttttgcaaaccaccaggtacataagctacggcgctcttcaaaggaacaccgttccctgctcccgccgtggtgcttcctattacaattgccagcctggagctcaggctaacccttacaaccgtggatgcagccgtatcacaaggtgcaggggc

SEQ ID NO: 25 - DNA, 330 bp, Populus tremula x, populus tremuloides
atggtgatgggcttgccatccaccgttcaaggaaatggggaccaccaccaccacctggggtggattccgaccaccacaaccaccagatcatcaatctgcgacaaggggtctttagcagagtgcatggctgaggaggatggggaagagtttgggatggacacggagatcaacaggcgcattttagcaactagcagttacgtcagctacggtgcgcttcagaagaacaatgttccttgctccaggcgtggtgcttcttactataattgcaagaatggtgctcaggctaatccttatagtcgtggatgcagtcgcattacaaggtgccggggt

SEQ ID NO: 26 - DNA, 360 bp, Arabidopsis thaliana
atggacaagtcctttactctgttcttaactcttacgattctcgtcgtcttcatcatctcttcacctccggtccaagccggcttcgccaacgacctcggtggtgtagcatgggctacgactggagacaatggttcaggctgccacggttcaatagcagagtgtatcggggcggaggaagaagaaatggactcagagatcaatagaagaatattggcgaccacaaaatacataagctatcagtctttgaaacggaacagtgtgccttgttcaagaagaggtgcgtcttattacaattgtcagaacggagctcaggctaatccttatagtcgtggttgcagcaaaattgctcgttgcaggagt

SEQ ID NO: 27 - DNA, 387 bp, Mesembryanthemum cystallinum
atggccgacactactacttctactagaaagctcctcttaatcctagccgtcggattcttcatcatcagcttcatcctgaccgccgatgcttccaccggcggcgacttcgaggcggcggcgggccagatgggctggatccgggccggatcgggctcgggctgcaagggagggagcgttggagagtgcttagggttcgatgaggagttcgagatggactcagatattcacaggcgcattttagcgacgaacagctacatcagctatggtgcgctgaacaagaacagagttccatgttcgaggcgtggggcctcttactacaactgccggcctggggcccaggctaacccttacagccgtggctgcagccgcatcactagatgcagaccc

SEQ ID NO: 28 - DNA, 354 bp, Glycine max
atggggagctcaaccttctgtgccttcttcctcctgtgtgcaatcttggccgtccacgtggcacaatcctcatcctccaccctcgacctggacgccttcttcctccctctcaagtccggctgtcgcggatccgtcgccgagtgcagcctcctcgccggcgacgacgccgagtttctgatggagtcggagagcaaccggcgcattctagcaggaaggagctacataagctacggtgcgctgaggaggaacactgtcccttgctcccgacgtggtgcttcctattacaattgccgccccggcgctcaggccaacccctacagccgcggatgcagtgctatcacccgatgcaggcgt

SEQ ID NO: 29 - DNA, 393 bp, Oryza sativa
atggcggcggcggcagtagcgttgctcgcgttcctcctcgccgcggcgtcagcggcgtcgtcggcgtcggcggcggcggcgacgctggtggagggcggcgtggtggggcgcgcggcggtggtgatgaggcggggagggaggacgtgccgtggcacggtgggggagtgcatggagttcctcggcgtcgacggcgagggggaggacgagctggcggcggcggcgacgggcaagcggcgggtgctgcagggcgggtcggggtacatcgggtacgacgcgctgcggcgcgacagcgtgccgtgctcgcagcgcggcgcgtcctactacaactgccagcccggcgccgaggccaacccttactcccgcggctgctccgccatcacccagtgcagggga

SEQ ID NO: 30 - DNA, 402 bp, Triticum aestivum
atggcgaagctgctggcggccggcgtcgcgttcctcctggccctcgccgcagcggcggcgctggccccgtctccggcctcggccttgaagggcgccggcgggctaggcctcggcggcgcggctgcggccgtggttatgcggcggggcgggcggacgtgccggggcacggtgggcgagtgcatggagtacttcggcgtggacggcgagggcgagagcgaggtggcggcgatggccggcaagcggcgggtgctgcaggacgggtccgggtacatcggctacgacgcgctgcggcgggacaacgtgccgtgctcgcagcgcggtgcctcctactacaactgccagcccggagccgaggccaacccctactcccgcggatgcagcgccatcacccagtgcaggggc

SEQ ID NO: 31 - DNA, 354 bp, Zea mays
atggctaagctcgcgctggcgctcctcctcctgctggccgtggcggcgtccgcgtcggccagcagcgggagccacctggatctcgacctgggcttcctctcctctggggaccgccggagggagtgccgcgggacggtggccgagtgcctcgccgaggagtcggatgaggagggccttgacctggccggatcccaccgccgcgcgctctacggcggcgggtacatcagctacggcgcgctgcggcgggacaacgtgccctgctcccgccgcggcgccagctactacaactgccgccccggcggccaggccaacccctaccaccgcggctgctcccgcatcacccgctgccgcggc

SEQ ID NO: 32 - DNA, 378 bp, Sorghum bicolor
atggctaggctcgcgctggcactcctgctgctgctagccgtggcggcgtcttcggcgtcggccacgggcgggagccaccacctggacctggacctgggcttcctctcctcctcctccggggcccgccggagggagtgccgcggcacggtggccgagtgcctcgccgcggaggagtctgaggaggaacgtctggacctggtctcctctcccgagtcccaccgccgcgcgctgtacggcaacgggtacatcagctacggcgcgctgcggcgggacaacgtgccctgctcccgccgcggcgccagctactacaactgccgccccggcggccaggccaacccctaccaccgcggctgctcccgcatcacccgctgccgcggc

SEQ ID NO: 33 - DNA, 366 bp, Hordeum vulgare
atggccggcgttaggagcctcgtgctgctcctcctagtgctcgccgtcgcggcgctgtccgcctccgccacctccatcgccggcggggaccacctgcagctcggcctcttcgccggccccggccgcggggagtgcaggggcaccgtggccgagtgcggcggggaggacgcggagggggagctcgggtcggcgtcggccgaggcgcaccgccgcgtgctgcagggccgcggctacatcagctacggcgcgctgcgcaggggcaccgtcccatgcaaccgccgcggtgccagctactacaactgccgccccggcgcccaggccaacccctaccaccgcggctgctcccgcatcacacgctgccgcggc

SEQ ID NO: 34 - DNA, 375 bp, Cryptomeria japonica
cgtggagatggtcgtgggttcgagactctgtgcgggtgccctgctcctgttcctggttttcttggcsstgtggatggctctttgggttctgccggtgctttgtggggcaatgcagatttcatgaaaatttcgaagaagacgacttgtgatgggtcgatcggtgattgttttccagaggaagaaatgttgatggattcggaactgaacaggaggattctggctacaacacagtatataagctatggagctctgagagcggattctgtgccctgcagtaagagtgggacatcttattataactgtgggagttctgggcaggccaatccttacagcaagtcttgtactcagattactcgctgtgcaagagatactagc

SEQ ID NO: 35 - DNA, 363 bp, Pinus taeda
atgggaaaggcgatctttatgttctccgccctgctgctgttctccgcgctgtccgcggctttaattggatctgctgccgcctcggagatggatgcgttgggggttctgtgggatctgaagccgcgatcccgctgtgagggttcgattggagaatgcttcgaggaagacgaaatgcagatggattccgagatccacaggcgttttctggccggccggacgtacatcagctacaagtcgttggcggccgacagcgtgccgtgttcgaagcgtggtacctcctactacaactgccgatccaccagccaggcgaatccttaccagcggagctgcacccagattactcgctgcgccagaagcacctcc

SEQ ID NO: 36 - protein, 27 aa, Medicago truncatula
ATTKYISYGALQRNTVPCSRRGASYYN

SEQ ID NO: 37 - protein, 17 aa, Lycopersicon esculentum
ATKKYISYGALQKNSVP

SEQ ID NO: 38 - DNA, 18 bp, Artificial sequence
  misc_feature: PCR primer used to amplify a tobacco RALF cDNA molecule
agtggtagctacgattgg

SEQ ID NO: 39 - DNA, 18 bp, Artificial sequence
  misc_feature: PCR primer used to amplify a tobacco RALF cDNA molecule
agagcatttcctcattcg

(19) United States
(12) Patent Application Publication
(10) Pub. No.: US 2003/0217389 A1
(43) Pub. Date: Nov. 20, 2003

(54) Novel peptides and methods of use

(76) Inventors: Clarence A Ryan, Pullman, WA (US); Gregory L Pearce, Palouse, WA (US); Daniel Scherer de Moura, Pullman, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/204,342

(22) PCT Filed: Feb. 15, 2001
(86) PCT No.: PCT/US01/05241

(51) Int. Cl. A01H 1/00; C12N 15/82; C07H 21/04; C07K 14/415

(52) U.S. Cl. 800/287; 435/419; 530/324; 536/23.6

(57) ABSTRACT

In one aspect, the present invention provides isolated RALF polypeptides that are useful for stimulating the growth of plant meristem cells. In another aspect, the present invention provides isolated nucleic acid molecules that are at least 90% identical to a nucleic acid molecule (SEQ ID NO: 2) that encodes a tobacco RALF precursor polypeptide (SEQ ID NO: 3). In a further aspect, the present invention provides isolated polypeptides that are at least 90% identical to the amino acid sequence of a tobacco RALF precursor polypeptide (SEQ ID NO: 3). In another aspect, the present invention provides vectors and plant cells comprising a vector of the invention. In other aspects, the present invention provides methods of inhibiting meristem growth in a plant, and methods of enhancing meristem growth in a plant.

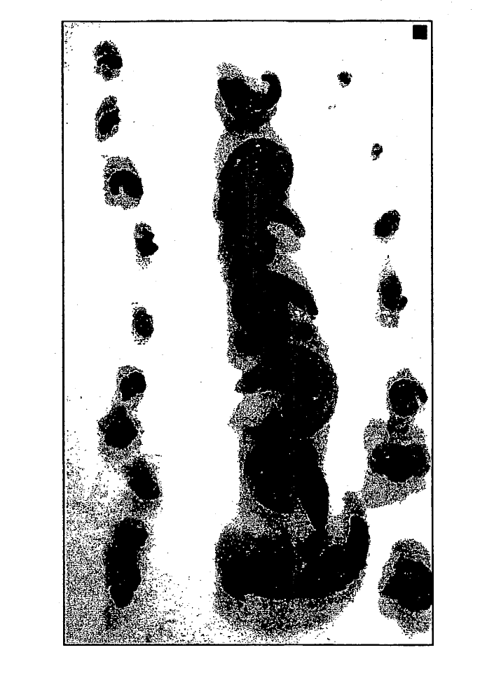

### FIELD OF THE INVENTION

The present invention relates to compositions and methods for stimulating or inhibiting the growth of plant meristems.

### BACKGROUND OF THE INVENTION

As the population of the world increases, there will be a greater demand for agricultural crops as food. Moreover, plants are increasingly being bred or genetically manipulated to produce useful chemical products, such as biologically active polypeptides. The yield of edible material from a crop plant, and the yield of one or more desired chemical products produced by a plant, depends, in part, on the size of the plant. The size of a plant is determined, at least in part, by the rate of growth of the plant meristems. Thus, there is a need for compositions and methods that promote the growth of plant meristems, thereby increasing the size and yield of the plant.

### SUMMARY OF THE INVENTION

In one aspect the present invention provides isolated polypeptides that consist of the amino acid sequence:

X₁X₂X₃X₄YX₅X₆YX₇X₈X₉X₁₀X₁₁X₁₂X₁₃X₁₄PCX₁₅X₁₆X₁₇GX₁₈SYYNC X₁₉X₂₀X₂₁X₂₂X₂₃ANPYX₂₄X₂₅X₂₆CX₂₇X₂₈IX₂₉X₃₀CX₃₁X₃₂ (SEQ ID NO:1) wherein X₁ through X₃₂ are as defined herein. The isolated polypeptides of this aspect of the invention are called RALF polypeptides and are useful for stimulating the growth of plant meristem cells.

In another aspect, the present invention provides isolated nucleic acid molecules that are at least 90% identical (such as at least 95% identical, or at least 99% identical) to a nucleic acid molecule consisting of the nucleic acid sequence set forth in SEQ ID NO: 2. SEQ ID NO: 2 sets forth the nucleic acid sequence of a tobacco cDNA molecule encoding a tobacco RALF precursor polypeptide (SEQ ID NO: 3).

In a further aspect, the present invention provides isolated polypeptides that are at least 90% identical (such as at least 95% identical, or at least 99% identical) to a polypeptide consisting of the amino acid sequence set forth in SEQ ID NO: 3. SEQ ID NO: 3 shows the amino acid sequence of a tobacco RALF precursor polypeptide.

In another aspect, the present invention provides vectors that include a nucleic acid molecule of the invention. In yet another aspect, the present invention provides plant cells, and plants, comprising a vector of the invention.

In another aspect, the present invention provides methods of inhibiting meristem growth in a plant, and methods of enhancing meristem growth in a plant.

The isolated polypeptides of the invention are useful, for example, to stimulate, and otherwise enhance, the growth and/or development of plant meristems in cultured plant cells or tissue, or in explants of plant material. Nucleic acid molecules encoding the isolated polypeptides of the invention can be introduced into, and expressed within, plants thereby stimulating, or otherwise enhancing, the growth and/or development of plant meristems. In antisense orientation within an expression vector, the isolated nucleic acid molecules of the invention can be used, for example, to inhibit the production of a RALF polypeptide through antisense inhibition. The vectors of the invention are useful, for example, in the methods of the invention for inhibiting or enhancing plant meristem growth.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

The FIGURE shows a photograph of excised tomato apical meristems cultured in the presence (top two rows) of a tomato RALF polypeptide (SEQ ID NO:4), or cultured in the presence (bottom row) of a control peptide that did not possess RALF activity. The top left portion of the FIGURE is indicated by a shaded square.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. (1989) Molecular Cloning: A Laboratory Manual, 2^{nd} ed., Cold Spring Harbor Press, Plainsview, N.Y. (1989), and Ausubel et al., Current Protocols in Molecular Biology (Supplement 47), John Wiley & Sons, New York (1999), for definitions and terns of the art.

Amino acid abbreviations used herein are set forth in Table 1 below.

TABLE 1

| | Three-letter | One-letter |
|---|---|---|
| Amino Acid | abbreviation | symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “isolated” used with respect to a nucleic acid molecule or polypeptide of the invention means a molecule that is substantially free from cellular components that are associated with the nucleic acid molecule or polypeptide as it is found in nature. As used in this context, the term “substantially free from cellular components” means that the nucleic acid molecule or polypeptide is purified to a purity level of greater than 80% (such as greater than 90%, greater than 95%, or greater than 99%). Moreover, the terms “isolated nucleic acid molecule” and “isolated polypeptide” include nucleic acid molecules and polypeptides which do not naturally occur, and have been produced by synthetic means. An isolated nucleic acid molecule or polypeptide generally resolves as a single, predominant, band by gel electrophoresis, and yields a nucleic acid or amino acid sequence profile consistent with the presence of a predominant nucleic acid molecule or polypeptide.

The term “RALF polypeptide” refers to a polypeptide that possesses the ability to stimulate the growth of at least one type of plant meristem (e.g., an apical meristem).

The term “percent identity” or “percent identical” when used in connection with the nucleic acid molecules and polypeptides of the present invention, is defined as the percentage of nucleic acid residues in a candidate nucleic acid sequence, or the percentage of amino acid residues in a candidate polypeptide sequence, that are identical with a subject nucleic acid sequence or polypeptide molecule sequence (such as the polypeptide amino acid sequence of SEQ ID NO:2), after aligning the candidate and subject sequences to achieve the maximum percent identity, and not considering any nucleic acid residue substitutions as part of the nucleic acid sequence identity. When making the comparison, the candidate nucleic acid sequence or polypeptide sequence (which may be a portion of a larger nucleic acid sequence or polypeptide sequence) is the same length as the subject nucleic acid sequence or polypeptide sequence, and no gaps are introduced into the candidate polynucleotide sequence or polypeptide sequence in order to achieve the best alignment.

Nucleic acid sequence identity can be determined in the following manner. The subject nucleic acid sequence is used to search a nucleic acid sequence database, such as the GenBank database (accessible at web site http://www.ncbi.nln.nih.gov/blast/), using the program BLASTN version 2.1 (based on Altschul et al., Nucleic Acids Research 25:3389-3402 (1997)). The program is used in the ungapped mode. Default filtering is used to remove sequence homologies due to regions of low complexity. The default parameters of BLASTN are utilized.

Amino acid sequence identity can be determined in the following manner. The subject polypeptide sequence is used to search a polypeptide sequence database, such as the GenBank database (accessible at web site http://www.ncbi.nln.nih.gov/blast/), using the BLASTP program. The program is used in the ungapped mode. Default filtering is used to remove sequence homologies due to regions of low complexity. The default parameters of BLASTP are utilized. Filtering for sequences of low complexity utilize the SEG program.

The term “hybridize under stringent conditions”, and grammatical equivalents thereof, refers to the ability of a nucleic acid molecule to hybridize to a target nucleic acid molecule (such as a target nucleic acid molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to nucleic acid molecules greater than about 100 bases in length, typical stringent hybridization conditions are no more than 25° C. to 30° C. (for example, 10° C.) below the melting temperature (Tm) of the native duplex (see generally, Sambrook et al., Molecular Cloning: A Laboratory Manual, 2nd Ed. Cold Spring Harbor Press, 1987; Ausubel et al., Current Protocols in Molecular Biology, Greene Publishing, 1987). Tm for nucleic acid molecules greater than about 100 bases can be calculated by the formula Tm=81.5+0.41% (G+C−log (Na⁺).

With respect to nucleic acid molecules having a length less than 100 bases, exemplary stringent hybridization conditions are 5° to 10° C. below Tm. On average, the Tm of a nucleic acid molecule of length less than 100 bp is reduced by approximately (500/oligonucleotide length)° C.

The term “vector” refers to a nucleic acid molecule, usually double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing the insert nucleic acid molecule, and, optionally, translating the transcript into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. The term “vector” includes the T-DNA of a Ti plasmid.

The term “expression vector” refers to a vector that includes the necessary elements that permit transcribing the insert nucleic acid molecule, and, optionally, translating the transcript into a polypeptide.

The term “meristem” refers to formative plant tissue composed of undifferentiated cells capable of dividing and giving rise to other meristem cells as well as to specialized cell types. Meristems occur at the growing points of plants (e.g., at the root tip, and at the apex of the aerial part of the plant).

In one aspect the present invention provides isolated polypeptides that consist of the amino acid sequence:

X₁X₂X₃X₄YX₅X₆YX₇X₈X₉X₁₀X₁₁X₁₂X₁₃X₁₄PCX₁₅X₁₆X₁₇GX₁₈SYYNC X₁₉X₂₀X₂₁X₂₂X₂₃ANPYX₂₄X₂₅X₂₆CX₂₇X₂₈IX₂₉X₃₀CX₃₁X₃₂ (SEQ ID NO:1) wherein: X₁ is A,Q,G,D,Y,R, or M; X₂ is T,G,D,Q, or R; X₃ is K,T,S,N,R, or G; X₄ is K,R,S,G,Q,T,N, or Y; X₅ is I or V; X₆ is S or G; X₇ is G,Q,D,K, or E; X₈ is A,S, or T; X₉ is L or M; X₁₀ is Q,K,N,R,A, or S; X₁₁ is K,R, or A; X₁₂ is N,D, or G; X₁₃ is S,T,N,R, or M; X₁₄ is V or I; X₁₅ is S or N; X₁₆ is R,Q, or K; X₁₇ is R or S; X₁₈ is A or T; X₁₉ is K,R,Q, or G; X₂₀ is P,N, or S; X₂₁ is G,S, or T; X₂₂ is A,G, or S; X₂₃ is Q or E; X₂₄ is T,S,N,H, or Q; X₂₅ is R or K; X₂₆ is G or S; X₂₇ is S or T; X₂₈ is A,R,K, or Q; X₂₉ is T or A; X₃₀ is R or Q; X₃₁ is R or A; and X₃₂ is S,G,P, or R.

As described in Example 1, the inventors utilized an assay, that identifies signaling molecules by their ability to cause a change in pH in a liquid plant cell culture, to identify and isolate an approximately 5 kDa polypeptide from tobacco. This polypeptide was called a RALF polypeptide (i.e., Rapid ALkalinization Factor) because it rapidly induced alkalinization of the liquid plant cell culture. The inventors obtained amino acid sequence (NH-ATKKYISYGALQKNSVP-COOH) (SEQ ID NO: 5) from the N-terminus of the 5 kDa polypeptide and used that sequence to search the NCBI sequence databases (accessible at http://www.ncbi.nlm.nih.gov/). This search identified a partial-length tomato (Lycopersicon esculentum) cDNA clone (Gen. Bank Accession No. AI781543) that included a region of amino acid sequence that was similar to the N-terminus sequence (SEQ ID NO: 5) of the tobacco RALF polypeptide.

The inventors chemically synthesized the portion of the tomato polypeptide sequence extending from (and including) the region similar to the tobacco 5 kDa polypeptide N-terminal sequence (SEQ ID NO: 5) through the carboxyl terminus of the tomato polypeptide. The amino acid sequence of the synthesized tomato polypeptide (called a tomato RALF polypeptide) is set forth in SEQ ID NO: 4. The tomato RALF polypeptide (SEQ ID NO: 4) was shown to induce alkalinization of a liquid tomato cell culture. A search of the NCBI databases revealed additional, partial-length, cDNA clones that encoded polypeptides (also called RALF polypeptides herein) that exhibit amino acid similarity to the amino acid sequence of the tomato RALF polypeptide (SEQ ID NO: 4).

These partial-length cDNA molecules were identified in the following plant species (numbers in parentheses are GenBank Accession numbers for the partial-length cDNA clones): pea (Pisum sativum, AA430937); alfalfa (Medicago truncatula, BE941609); cotton (Gossypium hirsutum, AI728208); poplar (Populus tremula x, populus tremuloides, AI163551); Arabidopsis (Arabidopsis thaliana, AAF02876, AV549237, RZ05b09R); ice plant (Mesembryanthemum crystallinum, BE033940); soy bean (Glycine mex BF424405); rice (Oryza sativa, AU077641); wheat (Triticum aestivum, BF483351); maize (Zea mays AI711894); sorghum (Sorghum bicolor BE363221); barley (Hordeum vulgare, AW925502); Cryptomeria (Cryptomeria japonica, AW084003); and pine (Pinus taeda, AI812921). The amino acid sequences of the RALF polypeptides from the foregoing plant species (including tomato) are shown in Table 2 below. The tobacco RALF sequence (SEQ ID NO:6) set forth in Table 2 was not identified in the database search, but was obtained by isolating and sequencing a tobacco RALF cDNA (SEQ ID NO:2) as described in Example 2. Thus, in one aspect, the present invention provides the isolated RALF polypeptides set forth in Table 2.

TABLE 2
Amino acid sequences of RALF polypeptides from various plant species.

| Plant species | | SEQ ID NO: |
|---|---|---|
| Tomato | ATKKYISYGALQKNSVPCSRRGASYYNCKPGAQANPYTRGCSAITRCRS | 4 |
| Tobacco | ATKKYISYGALQKNSVPCSRRGASYYNCKPGAQANPYSRGCSAITRCRS | 6 |
| Pea | ATTKYISYGALQRNTVPCSRRGASYYNCRPGAQANPYSRGCSAITRCRS | 7 |
| Medicago | ATTKYISYGALQRNTVPCSRRGASYYNCRPGAQANPYSRGCSAITRCRG | 8 |
| Cotton | QTTRYISYGALQRNTVPCSRRGASYYNCQPGAQANPYNRGCSRITRCRG | 9 |
| Poplar | ATSSYVSYGALQKNNVPCSRRGASYYNCKNGAQANPYSRGCSRITRCRG | 10 |
| Arabidopsis | ATTKYISYQSLKRNSVPCSRRGASYYNCQNGAQANPYSRGCSKIARCRS | 11 |
| Ice Plant | ATNSYISYGALNKNRVPCSRRGASYYNCRPGAQANPYSRGCSRITRCRP | 12 |
| Soybean | AGRSYISYGALRRNTVPCSRRGASYYNCRPGAQANPYSRGCSAITRCRR | 13 |
| Rice | GGSGYIGYGALRRDSVPCSQRGASYYNCQPGAEANPYSRGCSAITQCRG | 14 |
| Wheat | DGSGYIGYGALRRDNVPCSQRGASYYNCQPGAEANPYSRGCSAITQCRG | 15 |
| Maize | YGGGYISYGALRRDNVPCSRRGASYYNCRPGGQANPYHRGCSRITRCRG | 16 |
| Sorghum | YGNGYISYGALRRDNVPCSRRGASYYNCRPGGQANPYHRGCSRITRCRG | 17 |
| Barley | QGRGYISYGALRRGTVPCNRRGASYYNCRPGAQANPYHRGCSRITRCRG | 18 |
| Cryptomeria | ATTQYISYGALRADSVPCSKSGTSYYNCGSSGQANPYSKSCTQITRCAR | 19 |
| Pine | AGRTYISYKSLAADSVPCSKRGTSYYNCRSTSQANPYQRSCTQITRCAR | 20 |

The inventors demonstrated that the tomato RALF polypeptide (SEQ ID NO: 4) stimulates the growth of tomato plant meristem tissue, as described in Example 3. In the experiments described in Example 3, the plant meristem tissue grew at least twice as fast in the presence of tomato RALF polypeptide (SEQ ID NO: 4) than in the absence of tomato RALF polypeptide (SEQ ID NO: 4).

The polypeptides of this aspect of the invention can be prepared, for example, using peptide synthesis methods that are well known in the art. Direct peptide synthesis using solid-phase techniques (see, e.g., Stewart et al., Solid-Phase Peptide Synthesis, W.H. Freeman Co., San Francisco Calif. (1969); Merrifield, J. Am. Chem. Soc. 85:2149-2154 (1963). Automated synthesis may be achieved, for example, using Applied Biosystems 431A Peptide Synthesizer (Foster City, Calif.) in accordance with the instructions provided by the manufacturer. Additionally the polypeptide sequences, or any fragment thereof, may be mutated during direct synthesis and, if desired, combined using chemical methods with other amino acid sequences.

Polypeptides of the invention can also be prepared, for example, by expressing nucleic acid molecules encoding the desired polypeptide(s) in a suitable host cell, such as E. coli. By way of representative example, a nucleic acid molecule (such as a cDNA molecule) encoding a polypeptide of the invention is cloned into a plasmid vector, such as a Bluescript plasmid (available from Stratagene, Inc., La Jolla, Calif.). The recombinant vector is then introduced into an E. coli strain (such as E. coli XL1-Blue, also available from Stratagene, Inc.) and the polypeptide encoded by the nucleic acid molecule is expressed in E. coli and then purified. For example, E. coli XL1-Blue harboring a Bluescript vector including a cDNA molecule of interest is grown overnight at 37° C. in LB medium containing 100 μg ampicillin/ml. A 50 μl aliquot of the overnight culture is used to inoculate 5 ml of fresh LB medium containing ampicillin, and the culture grown at 37° C. with vigorous agitation to A₆₀₀=0.5 before induction with 1 mM IPTG. After an additional two hours of growth, the suspension is centrifuged (1000× g, 15 min, 4° C.), the media removed, and the pelleted cells resuspended in 1 ml of cold buffer that preferably contains 1 mM EDTA and one or more proteinase inhibitors. The cells can be disrupted by sonication with a microprobe. The chilled sonicate is cleared by centrifugation and the expressed, recombinant polypeptide purified from the supernatant by art-recognized protein purification techniques, such as those described herein.

Representative examples of art-recognized techniques for purifying, or partially purifying, polypeptides from biological material, such as from prokaryotic cells that express the desired polypeptide(s), are: exclusion chromatography, ion-exchange chromatography, hydrophobic interaction chromatography, reversed-phase chromatography and immobilized metal affinity chromatography.

Hydrophobic interaction chromatography and reversed-phase chromatography are two separation methods based on the interactions between the hydrophobic moieties of a sample and an insoluble, immobilized hydrophobic group present on the chromatography matrix. In hydrophobic interaction chromatography the matrix is hydrophilic and is substituted with short-chain phenyl or octyl nonpolar groups. The mobile phase is usually an aqueous salt solution. In reversed phase chromatography the matrix is silica that has been substituted with longer n-alkyl chains, usually C₈ (octylsilyl) or C₁₈ (octadecylsilyl). The matrix is less polar than the mobile phase. The mobile phase is usually a mixture of water and a less polar organic modifier.

Separations on hydrophobic interaction chromatography matrices are usually done in aqueous salt solutions, which generally are nondenaturing conditions. Samples are loaded onto the matrix in a high-salt buffer and elution is by a descending salt gradient. Separations on reversed-phase media are usually done in mixtures of aqueous and organic solvents, which are often denaturing conditions. In the case of polypeptide and/or peptide purification, hydrophobic interaction chromatography depends on surface hydrophobic groups and is carried out under conditions which maintain the integrity of the polypeptide molecule. Reversed-phase chromatography depends on the native hydrophobicity of the polypeptide and is carried out under conditions which expose nearly all hydrophobic groups to the matrix, i.e., denaturing conditions.

Ion-exchange chromatography is designed specifically for the separation of ionic or ionizable compounds. The stationary phase (column matrix material) carries ionizable functional groups, fixed by chemical bonding to the stationary phase. These fixed charges carry a counterion of opposite sign. This counterion is not fixed and can be displaced. Ion-exchange chromatography is named on the basis of the sign of the displaceable charges. Thus, in anion ion-exchange chromatography the fixed charges are positive and in cation ion-exchange chromatography the fixed charges are negative.

Retention of a molecule on an ion-exchange chromatography column involves an electrostatic interaction between the fixed charges and those of the molecule, binding involves replacement of the nonfixed ions by the molecule. Elution, in turn, involves displacement of the molecule from the fixed charges by a new counterion with a greater affinity for the fixed charges than the molecule, and which then becomes the new, nonfixed ion.

The ability of counterions (salts) to displace molecules bound to fixed charges is a function of the difference in affinities between the fixed charges and the nonfixed charges of both the molecule and the salt. Affinities in turn are affected by several variables, including the magnitude of the net charge of the molecule and the concentration and type of salt used for displacement.

Solid-phase packings used in ion-exchange chromatography include cellulose, dextrans, agarose, and polystyrene. The exchange groups used include DEAE (diethylaminoethyl), a weak base, that will have a net positive charge when ionized and will therefore bind and exchange anions; and CM (carboxymethyl), a weak acid, with a negative charge when ionized that will bind and exchange cations. Another form of weak anion exchanger contains the PEI (polyethyleneimine) functional group. This material, most usually found on thin layer sheets, is useful for binding polypeptides at pH values above their pI. The polystyrene matrix can be obtained with quaternary ammonium functional groups for strong base anion exchange or with sulfonic acid functional groups for strong acid cation exchange. Intermediate and weak ion-exchange materials are also available. Ion-exchange chromatography need not be performed using a column, and can be performed as batch ion-exchange chromatography with the slurry of the stationary phase in a vessel such as a beaker.

Gel filtration is performed using porous beads as the chromatographic support. A column constructed from such beads will have two measurable liquid volumes, the external volume, consisting of the liquid between the beads, and the internal volume, consisting of the liquid within the pores of the beads. Large molecules will equilibrate only with the external volume while small molecules will equilibrate with both the external and internal volumes. A mixture of molecules (such as proteins) is applied in a discrete volume or zone at the top of a gel filtration column and allowed to percolate through the column. The large molecules are excluded from the internal volume and therefore emerge first from the column while the smaller molecules, which can access the internal volume, emerge later. The volume of a conventional matrix used for protein purification is typically 30 to 100 times the volume of the sample to be fractionated. The absorbance of the column effluent can be continuously monitored at a desired wavelength using a flow monitor.

A technique that is often applied to the purification of polypeptides is High Performance Liquid Chromatography (HPLC). HPLC is an advancement in both the operational theory and fabrication of traditional chromatographic systems. HPLC systems for the separation of biological macromolecules vary from the traditional column chromatographic systems in three ways; (1) the column packing materials are of much greater mechanical strength, (2) the particle size of the column packing materials has been decreased 5- to 10-fold to enhance adsorption-desorption kinetics and diminish bandspreading, and (3) the columns are operated at 10-60 times higher mobile-phase velocity. Thus, by way of non-limiting example, HPLC can utilize exclusion chromatography, ion-exchange chromatography, hydrophobic interaction chromatography, reversed-phase chromatography and immobilized metal affinity chromatography. Art-recognized techniques for the purification of proteins and peptides are set forth in Methods in Enzymology, Vol. 182, Guide to Protein Purification, Murray P. Deutscher, ed. (1990).

In another aspect, the present invention provides isolated nucleic acid molecules that are at least 90% identical (such as at least 95% identical, or at least 99% identical) to a nucleic acid molecule consisting of the nucleic acid sequence set forth in SEQ ID NO: 2. SEQ ID NO: 2 shows the nucleic acid sequence of a tobacco cDNA molecule encoding a tobacco RALF precursor polypeptide (SEQ ID NO: 3). The isolation of the tobacco RALF cDNA molecule (SEQ ID NO: 2) is described in Example 2. The nucleic acid molecules of this aspect of the invention can be isolated by using a variety of cloning techniques known to those of ordinary skill in the art. For example, all, or portions of, the tobacco RALF cDNA molecule having the sequence set forth in SEQ ID NO:2 can be used as a hybridization probe to screen a plant genomic or cDNA library. The technique of hybridizing radiolabelled nucleic acid probes to nucleic acids immobilized on nitrocellulose filters or nylon membranes can be used to screen the genomic or cDNA library. Exemplary hybridization and wash conditions are: hybridization for 20 hours at 65° C. in 5.0×SSC, 0.5% sodium dodecyl sulfate, 1× Denhardt's solution; washing (three washes of twenty minutes each at 55° C.) in 1.0×SSC, 1%(w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

Again, by way of example, nucleic acid molecules of this aspect of the invention can be isolated by the polymerase chain reaction (PCR) described in The Polymerase Chain Reaction (K. B. Mullis et al., eds. 1994), incorporated herein by reference. Gobinda et al. (PCR Methods Applic. 2:318-22 (1993)), incorporated herein by reference, disclose “restriction-site PCR” as a direct method which uses universal primers to retrieve unknown sequence adjacent to a known locus. First, genomic DNA is amplified in the presence of a linker-primer, that is homologous to a linker sequence ligated to the ends of the genomic DNA fragments, and in the presence of a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Further, by way of example, inverse PCR permits acquisition of unknown sequences starting with primers based on a known region (Triglia, T. et al., Nucleic Acids Res 16:8186 (1988), incorporated herein by reference). The method uses several restriction enzymes to generate a suitable fragment in the known region of a gene. The fragment is then circularized by intramolecular ligation and used as a PCR template. Divergent primers are designed from the known region.

Typically, the nucleic acid sequence of a primer useful to amplify nucleic acid molecules of the invention by PCR is based on a conserved region of amino acid sequence of the RALF polypeptides of the invention (such as the RALF polypeptides having the amino acid sequences set forth in Table 2).

In a further aspect, the present invention provides isolated polypeptides that are at least 90% identical (such as at least 95% identical, or at least 99% identical) to a polypeptide consisting of the amino acid sequence set forth in SEQ ID NO: 3. SEQ ID NO: 3 shows the amino acid sequence of a tobacco RALF precursor polypeptide. The polypeptides of this aspect of the invention can, for example, be chemically synthesized as described supra, or can be produced, for example, by expressing a nucleic acid molecule of the invention in an appropriate host cell (such as a prokaryotic host cell) and purifying the polypeptide therefrom.

In another aspect, the present invention provides vectors that include a nucleic acid molecule of the invention. In one embodiment of this aspect of the invention, the present invention provides vectors based on the Ti plasmid of Agrobacterium species.

In yet another aspect, the present invention provides plant cells, and plants, comprising a vector of the invention. The vectors can be introduced into the genome of plant cells using techniques well known to those skilled in the art. These methods include, but are not limited to, (1) direct DNA uptake, such as particle bombardment or electroporation (see, Klein et al., Nature 327:70-73 (1987); U.S. Pat. No. 4,945,050), and (2) Agrobacterium-mediated transformation (see, e.g., U.S. Pat. Nos: 6,051,757; 5,731,179; 4,693,976; 4,940,838; 5,464,763; and 5,149,645). Within the cell, the transgenic sequences may be incorporated within the chromosome. The skilled artisan will recognize that different independent insertion events may result in different levels and patterns of gene expression (Jones et al., EMBO J. 4:2411-2418 (1985); De Almeida et al., MGG 218:78-86 (1989)), and thus that multiple events may have to be screened in order to obtain lines displaying the desired expression level and pattern.

Transgenic plants can be obtained, for example, by transferring vectors that include a selectable marker gene, e.g., the kan gene encoding resistance to kanamycin, into Agrobacterium tumifaciens containing a helper Ti plasmid as described in Hoeckema et al., Nature, 303:179-181 (1983) and culturing the Agrobacterium cells with leaf slices, or other tissues or cells, of the plant to be transformed as described by An et al., Plant Physiology, 81:301-305 (1986). Transformation of cultured plant host cells is normally accomplished through Agrobacterium tumifaciens.

Transformed plant calli may be selected through the selectable marker by growing the cells on a medium containing, for example, kanamycin, and appropriate amounts of phytohormone such as naphthalene acetic acid and benzyladenine for callus and shoot induction. The plant cells may then be regenerated and the resulting plants transferred to soil using techniques well known to those skilled in the art.

In addition to the methods described above, several methods are known in the art for transferring cloned DNA into a wide variety of plant species, including gymnosperms, angiosperms, monocots and dicots (see, e.g., Glick and Thompson, eds., Methods in Plant Molecular Biology, CRC Press, Boca Raton, Fla. (1993), incorporated by reference herein). Representative examples include electroporation-facilitated DNA uptake by protoplasts in which an electrical pulse transiently permeabilizes cell membranes, permitting the uptake of a variety of biological molecules, including recombinant DNA (see, e.g., Rhodes et al., Science, 240:204-207 (1988)); treatment of protoplasts with polyethylene glycol (see, e.g., Lyznik et al., Plant Molecular Biology, 13:151-161 (1989)); and bombardment of cells with DNA-laden microprojectiles which are propelled by explosive force or compressed gas to penetrate the cell wall (see, e.g., Klein et al., Plant Physiol. 91:440-444 (1989) and Boynton et al., Science, 240(4858):1534-1538 (1988)). A method that has been applied to Rye plants (Secale cereale) is to directly inject plasmid DNA, including a selectable marker gene, into developing floral tillers (de la Pena et al., Nature 325:274-276 (1987)). Further, plant viruses can be used as vectors to transfer genes to plant cells. Examples of plant viruses that can be used as vectors to transform plants include the Cauliflower Mosaic Virus (see, e.g., Brisson et al., Nature 310:511-514 (1984); Other useful techniques include: site-specific recombination using the Cre-lox system (see, U.S. Pat. No. 5,635,381); and insertion into a target sequence by homologous recombination (see, U.S. Pat. No. 5,501,967). Additionally, plant transformation strategies and techniques are reviewed in Birch, R. G., Ann Rev Plant Phys Plant Mol Biol., 48:297 (1997); Forester et al., Exp. Agric., 33:15-33 (1997).

Positive selection markers may also be utilized to identify plant cells that include a vector of the invention. For example, U.S. Pat. Nos. 5,994,629, 5,767,378, and 5,599,670, describe the use of a beta-glucuronidase transgene and application of cytokinin-glucuronide for selection, and use of mannophosphatase or phosphmanno-isomerase transgene and application of mannose for selection.

The cells which have been transformed may be grown into plants by a variety of art-recognized means. See, for example, McConnick et al., Plant Cell Reports 5:81-84 (1986). These plants may then be grown, and either selfed or crossed with a different plant strain, and the resulting homozygotes or hybrids having the desired phenotypic characteristic identified. Two or more generations may be grown to ensure that the subject phenotypic characteristic is stably maintained and inherited and then seeds harvested to ensure the desired phenotype or other property has been achieved.

The following are representative plant species that are suitable for genetic manipulation in accordance with the present invention. The citations are to representative publications disclosing genetic transformation protocols that can be used to genetically transform the listed plant species. Rice (Alam, M. F. et al., Plant Cell Rep. 18:572-575 (1999)); maize (U.S. Pat. Nos. 5,177,010 and 5,981,840); wheat (Ortiz, J. P. A., et al., Plant Cell Rep. 15:877-881 (1996)); tomato U.S. Pat. No. 5,159,135); potato (Kumar, A., et al., Plant J. 9:821-829 (1996)); cassava (Li, H.-Q., et al., Nat. Biotechnology 14:736-740 (1996)); lettuce (Michelmore, R., et al., Plant Cell Rep. 6:439-442 (1987)); tobacco (Horsch, R. B., et al., Science 227:1229-1231 (1985)); cotton (U.S. Pat. Nos. 5,846,797 and 5,004,863); grasses (U.S. Pat. Nos. 5,187,073 and 6,020,539); peppermint (X. Niu et al., Plant Cell Rep. 17:165-171 (1998)); citrus plants (Pena, L. et al., Plant Sci. 104:183-191 (1995)); caraway (F. A. Krens, et al., Plant Cell Rep., 17:39-43 (1997)); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); and cereals (U.S. Pat. No. 6,074,877).

In another aspect, the present invention provides methods of inhibiting meristem growth in a plant, the methods comprising the steps of (a) introducing into a plant an expression vector that comprises a nucleic acid sequence that is transcriptionally expressed to yield a nucleic acid molecule (such as an RNA molecule) that hybridizes under stringent conditions to a nucleic acid molecule consisting of a nucleic acid sequence selected from the group of sequences consisting of SEQ ID NO: 2, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35, and (b) transcriptionally expressing the nucleic acid sequence in the plant.

SEQ ID NO: 2 discloses the nucleic acid sequence of a tobacco cDNA molecule, isolated by the present inventors, that encodes a RALF precursor polypeptide. SEQ ID NO: 21 through SEQ ID NO: 35 disclose portions of the nucleic acid sequences of partial-length cDNA molecules isolated from various plant species. These portions (SEQ ID NO: 21 through SEQ ID NO: 35) do not include sequence from the 5′- or 3′-untranslated regions of the cDNA molecules. The nucleic acid sequences of the partial-length cDNA molecules are available in the publicly-accessible GenBank database. Table 3 sets forth the GenBank accession numbers of the partial-length cDNA molecules which include the sequences set forth in SEQ ID NO: 21 through SEQ ID NO: 35, and the plant species from which the partial-length cDNA molecules were obtained.

TABLE 3

| SEQ ID NO. | GenBank Accession No. | Plant Species |
|---|---|---|
| 21 | AI781543 | Tomato |
| 22 | AA430937 | Pea |
| 23 | BE941609 | Medicago |
| 24 | AI728208 | Cotton |
| 25 | AI163551 | Poplar |
| 26 | AAF02876 | Arabidopsis |
| 27 | BE033940 | Mesembryanthemum |
| 28 | BF424405 | Soybean |
| 29 | AU077641 | Rice |
| 30 | BF483351 | Wheat |
| 31 | AI711894 | Maize |
| 32 | BE363221 | Sorgham |
| 33 | AW925502 | Barley |
| 34 | AW084003 | Cryptomeria |
| 35 | AI812921 | Pine |

Typically, though not necessarily, in the methods of inhibiting meristem growth in a plant, a nucleic acid molecule is utilized (typically by incorporation into an expression vector in antisense orientation relative to a promoter) that yields a transcriptional product that hybridizes under stringent conditions to one of the foregoing cDNA molecules (SEQ ID NO: 2 and SEQ ID NO: 21 through SEQ ID NO: 35) that was isolated from the same species of plant which is being treated in accordance with the invention to inhibit meristem growth. For example, to inhibit meristem growth in a tomato plant, typically a nucleic acid molecule is utilized that yields an RNA transcript that hybridizes under stringent conditions to the nucleic acid molecule consisting of the sequence set forth in SEQ ID NO: 21. Representative stringent hybridization conditions are 1.0×SSC at 60° C. for 20 minutes. Additional, representative, stringent hybridization conditions are 0.5×SSC at 60° C. for 20 minutes. The ability to hybridize under stringent hybridization conditions can be determined, for example, by initially hybridizing under less stringent conditions (e.g., 3×SSC at 50° C.), then increasing the stringency to the desired stringent conditions, for example to 1.0×SSC at 60° C. for 20 minutes, or to 0.5×SSC at 60° C. for 20 minutes.

The expression vectors of this aspect of the invention can be introduced into plant cells by any art-recognized means, such as the methods set forth supra. Plants can be regenerated from the genetically modified plant cells as described supra. The nucleic acid sequence is transcriptionally expressed to yield a nucleic acid molecule that hybridizes under stringent conditions to a nucleic acid molecule consisting of a nucleic acid sequence selected from the group of sequences consisting of SEQ ID NO: 2, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35. The nucleic acid sequence is an antisense molecule that is typically oriented in antisense orientation with respect to a constitutive (such as the CaMV 35S promoter) or inducible promoter within the vector.

An antisense nucleic acid molecule is a DNA sequence that is inverted relative to its normal orientation for transcription and so expresses an RNA transcript that is complementary to a target mRNA molecule expressed within the host cell (i.e., the RNA transcript of the antisense nucleic acid molecule can hybridize to the target mRNA molecule through Watson-Crick base pairing). An antisense nucleic acid molecule may be constructed in a number of different ways provided that it is capable of interfering with the expression of a target gene. The antisense nucleic acid molecule can be constructed by inverting the coding region (or a portion thereof) of the target gene relative to its normal orientation for transcription to allow the transcription of its complement, hence the RNAs encoded by the antisense and sense gene are complementary.

The antisense nucleic acid molecule generally will be substantially identical to at least a portion of the target gene or genes. The sequence, however, need not be perfectly identical to inhibit expression. Generally, higher homology can be used to compensate for the use of a shorter antisense nucleic acid molecule. The antisense nucleic acid molecule generally will be substantially identical (although in antisense orientation) to the target gene. The minimal identity will typically be greater than about 65%, but a higher identity might exert a more effective repression of expression of the endogenous sequences. Substantially greater identity of more than about 80% is preferred, though about 95% to absolute identity would be most preferred.

Furthermore, the antisense nucleic acid molecule need not have the same intron or exon pattern as the target gene, and non-coding segments of the target gene may be equally effective in achieving antisense suppression of target gene expression as coding segments. Normally, a DNA sequence of at least about 30 or 40 nucleotides should be used as the antisense nucleic acid molecule, although a longer sequence is preferable.

For example, antisense nucleic acid molecules can be utilized that produce RNA which hybridizes with mRNA encoding RALF polypeptides in a plant meristem. In this manner, the antisense RNA will prevent expression of the RALF gene(s) resulting in meristem growth inhibition.

Alternately, ribozymes can be utilized which target RALF mRNA in a plant meristem. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

Ribozymes useful in the practice of the invention typically comprise a hybridizing region of at least about nine nucleotides which is complementary in nucleotide sequence to at least part of the target RNA and a catalytic region which is adapted to cleave the target RNA (see, e.g., EPA No. 0 321 201; WO88/04300; Haseloff & Gerlach, Nature 334:585-591 (1988); Fedor & Uhlenbeck, Proc. Natl. Acad. Sci. USA 87:1668-1672 (1990); Cech & Bass, Ann. Rev. Biochem. 55:599-629 (1986)).

In another aspect, the present invention provides methods of enhancing meristem growth in a plant, the methods comprising the steps of (a) introducing into the plant an expression vector comprising a nucleic acid molecule that encodes a RALF polypeptide; and (b) expressing the RALF polypeptide within the plant. The RALF polypeptide stimulates, and so enhances, growth of the meristem. Some nucleic acid molecules encoding a RALF polypeptide are at least 70% identical (such as at least 80% identical, or at least 95% identical) to a nucleic acid molecule consisting of the nucleic acid sequence set forth in SEQ ID NO: 2. Some nucleic acid molecules encoding a RALF polypeptide hybridize under stringent conditions to the complement of a nucleic acid molecule consisting of a sequence selected from the group of sequences set forth in SEQ ID NO: 2, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

Representative stringent hybridization conditions are 1.0×SSC at 60° C. for 20 minutes. Additional, representative, stringent hybridization conditions are 0.5×SSC at 60° C. for 20 minutes. The ability to hybridize under stringent hybridization conditions can be determined, for example, by initially hybridizing under less stringent conditions (e.g., 3×SSC at 50° C.), then increasing the stringency to the desired stringent conditions, for example to 1.0×SSC at 60° C. for 20 minutes, or to 0.5×SSC at 60° C. for 20 minutes.

The expression vectors of this aspect of the invention can be introduced into plant cells by any art-recognized means, such as the methods, set forth supra. Plants can be regenerated from the genetically modified plant cells as described supra.

### Example 1

This example describes the isolation of a tobacco RALF polypeptide which was sequenced at the N-terminus to yield the amino acid sequence set forth in SEQ ID NO: 5.

Plant Cell Culture Alkalinization Assay: Tobacco suspension cells were maintained in MS media. The media was adjusted to pH 5.6 with KOH. Three ml aliquots of one week old cultures were transferred into 35 ml of media in 125 ml flasks and placed on an orbital shaker at 160 rpm in the dark for growth. Cells were used for assay 3-5 days after transfer (cell density was approximately 0.5 to 1.5×10⁶ cells/ml). One ml of cells was aliquoted into each well of 24-well cell culture cluster plates (Costar, number 3527, Corning Incorporated, Corning, N.Y.) and allowed to equilibrate on an orbital shaker at 160 rpm for 1 hr. Aliquots (1 to 10 μl) of fractions to be tested were added to the cells and the increasing pH change of the medium (medium alkalinization) was detected with time using an Orion Model EA940 pH meter with an Orion semi-micro pH electrode (Catalog number 8103BN, Orion, Beverly, Mass.).

Polypeptide Isolation: Tobacco plants were harvested for extraction four weeks after planting. Plants were harvested in liquid nitrogen and stored at −20° C. until use. Each preparation consisted of approximately 450 plants with a wet weight of about 1.1 kilograms (kgs). The leaves were homogenized in a 4 L Waring blender with 2.8 L of 1% trifluoroacetic acid (TFA) for 2 min and filtered through 8 layers of cheesecloth and one layer of Miracloth (Calbiochem, LaJolla, Calif.). The liquid was centrifuged at 10,000× g for 20 min.

The supernatant from the initial centrifugation was loaded onto a 40 μm, 3×25 cm C18 reversed phase flash column (Bakerbond, Catalog number 7025-00), previously equilibrated with 0.1% TFA/H₂O. Compressed nitrogen was used at 8 psi to elute the extract. After sample loading, the column was washed with 0.1% TFA/H₂O and the retained material was eluted with successive washes of 10, 30, and 50% methanol in 0.1% TFA. The 50% methanol-eluting fraction had strong activity in alkalinizing the media of tobacco suspension cells. The methanol was removed using a rotary evaporator, followed by lyophilization to dryness. Several preparations were collected and stored to provide a stock for further purification.

The lyophilized powder (40 mg dissolved in 1 ml of Solvent A, consisting of 0.1% TFA in water), was injected into a semipreparative reversed-phase C18 column (Vydac, Hesperia, Calif., Column 218TP510, 10×250 mm, 5 μm beads, 300A pores). Samples were injected in solvent A and after 2 minutes (min), a 90 min gradient from 0-40% Solvent B (0.1% TFA in acetonitrile) was employed. The flow rate was 2 ml/min, and eluted peaks were monitored at 225 nm. One-minute fractions were collected and the ability of the fractions to alkalinate tobacco suspension cells was determined as described above. The active peak eluting at 77 and 78 min from 8 runs (320 mg total) was pooled and lyophilized.

Strong cation exchange chromatography was then performed using the active peaks from the C18 semipreparative column on a polysulfoethyl aspartamide column (4.6×200 mm, 5 μm, the Nest Group, Southborough, Mass.) was employed with the use of the following solvent system: solvent A, 5 mM potassium phosphate, pH 3, in 25% acetonitrile; solvent B, 5 mM potassium phosphate, 500 mM potassium chloride, pH 3, in 25% acetonitrile. Active fractions were dissolved in 1 ml of solvent A, centrifuged, and applied to the column. After a 2 min wash with solvent A, a 60 min gradient was applied to 100% solvent B. The flow rate was 1 ml/min and the elution profile was monitored by absorbance at 214 nm. One minute fractions were collected and the alkalinating activity (fractions 50-51) was identified, pooled and lyophilized.

Narrow-bore reversed phase chromatography was performed on the active peak. The column (Vydac, Hesperia, Calif., Column 218 TP52, 2.1×250 mm, 5 mm) was equilibrated with solvent A consisting of 0.1% TFA in water and peaks were eluted with solvent B consisting of 0.05% TFA in methanol. After a 2 min wash, a 90 min gradient was applied to 100% solvent B. The flow rate was 0.25 ml/min and fractions were collected at 1 min intervals. Alkalinating activity was determined as described above using 2 μl of each fraction. Alkalinating activity was found between fractions 61-63 and this was used for amino acid sequence analysis and MALDI-MS analysis. The mass was determined to be 5338 Daltons and the amino-terminal sequence was ATKKYISYGALQKNSVP-(SEQ ID NO: 5).

This same basic method was used to obtain N-terminal sequence from alfalfa (NH-ATTKYISYGALQRNTVPCSRRGASYYN-COOH (SEQ ID NO: 36)) and tomato (NH-ATKKYISYGALQKNSVP-COOH (SEQ ID NO: 37)).

### Example 2

This example describes the cloning of a tobacco RALF cDNA molecule (SEQ ID NO: 2).

The N-terminal peptide sequence ATKKYISYGALQKNSVP (SEQ ID NO: 5) derived from tobacco was used to search NCBI databases for homologous proteins. Several translation products of tomato ESTs showed homology to the tobacco peptide (SEQ ID NO: 5). Two tomato EST specific primers (5′-AGTGGTAGCTACGATTGG-3′) (SEQ ID NO: 38) and (5′-AGAGCATTTCCTCATTCG-3′) (SEQ ID NO: 39) were designed and used in reverse transcriptase polymerase chain reactions, using total tomato RNA as template, that resulted in the amplification of a 427 bp fragment. The amplified fragment was labeled with [α-³²P]dCTP (NEN-Dupont Co, Wilmington, Del.) using the DECAprime II DNA labeling kit (Ambion, Austin, Tex.) and used to screen a tobacco leaf cDNA library. The library was constructed using leaf poly-A mRNA extracted from three-week-old tobacco plants using Trizol reagent (Life Technologies, Santa Clara, Calif.) and Poly(A) Quik mRNA isolation kit (Stratagene, Cedar Creek, Tex.). Tobacco cDNA was synthesized (ZAP Express cDNA synthesis Kit, Stratagene, Cedar Creek, Tex.) and cloned using the ZAP Express cDNA Gigapack III Gold Cloning Kit (Stratagene, Cedar Creek, Tex.). Isolated clones were sequenced and confirmed to contain the previously obtained tobacco N-terminal sequence (SEQ ID NO: 5) (bases at position 329 to 379). The nucleic acid sequence of a tobacco RALF cDNA is set forth in SEQ ID NO: 2.

### Example 3

This example shows the ability of a tomato RALF polypeptide (SEQ ID NO: 4) to enhance the growth of a tomato plant meristem.

Tomato plants (Lycopersicon esculentum cv. Castlemart) were grown in a growth chamber with 17 hours of light (300 μmol m⁻²s⁻¹) per day at 28° C. and seven hour nights at 18° C. Apical meristems were excised from shoot tips of two week old tomato plants. Shoot tips (1-2 cm long) were surface sterilized using 70% ethanol solution for 20-30 seconds, followed by a 20 minute incubation in a 1.5% (v/v) commercial bleach solution. Shoot tips were washed once in sterilized water after the ethanol treatment, and four times after bleaching. Apical meristems (including meristematic tissue, dome, with a leaf primordium) were carefully isolated under a stereoscope.

Isolated meristems were immediately inoculated into a liquid media (MS salts and vitamin mixture powder (Life Technologies) and 20 g/L of sucrose) containing either tomato RALF (100 nM) (SEQ ID NO: 4), or a control peptide (100 nM) that was not capable of stimulating meristem growth. Four meristems were inoculated in a 125 mL Erlenmeyer flask containing 10 mL of media, and flasks were gently shaken at 60 rpm. Sixteen meristems were tested for each treatment in four separate flasks. The liquid media was replaced with fresh media on the 5th, 12th and 14th days of the experiment. The experiment was evaluated and terminated on the 16th day. As shown in the FIGURE, tomato RALF (SEQ ID NO: 4) stimulated growth of the isolated apical meristems, compared to growth of the apical meristems treated with a control peptide.

While the preferred embodiment of the invention has been illustrated and described, it will be appreciated that various changes can be made therein without departing from the spirit and scope of the invention.

The embodiments of the invention in which an exclusive property of privilege is claimed are defined as follows:

1. An isolated polypeptide consisting of the amino acid sequence:
X₁X₂X₃X₄YX₅X₆YX₇X₈X₉X₁₀X₁₁X₁₂X₁₃X₁₄PCX₁₅X₁₆X₁₇GX₁₈SYYNC X₁₉X₂₀X₂₁X₂₂X₂₃ANPYX₂₄X₂₅X₂₆CX₂₇X₂₈IX₂₉X₃₀CX₃₁X₃₂, (SEQ ID NO: 1) wherein: X₁ is A,Q,G,D,Y,R, or M; X₂ is T,G,D,Q, or R; X₃ is K,T,S,N,R, or G; X₄ is K,R,S,G,Q,T,N, or Y; X₅ is I or V; X₆ is S or G; X₇ is G,Q,D,K, or E; X₈ is A,S, or T; X₉ is L or M; X₁₀ is Q,K,N,R,A, or S; X₁₁ is K,R, or A; X₁₂ is N,D, or G; X₁₃ is S,T,N,R, or M; X₁₄ is V or I; X₁₅ is S or N; X₁₆ is R,Q, or K; X₁₇ is R or S; X₁₈ is A or T; X₁₉ is K,R,Q, or G; X₂₀ is P,N, or S; X₂₁ is G,S, or T; X₂₂ is A,G, or S; X₂₃ is Q or E; X₂₄ is T,S,N,H, or Q; X₂₅ is R or K; X₂₆ is G or S; X₂₇ is S or T; X₂₈ is A,R,K, or Q; X₂₉ is T or A; X₃₀ is R or Q; X₃₁ is R or A; and X₃₂ is S,G,P, or R.

2. An isolated polypeptide of claim 1 consisting of the amino acid sequence set forth in SEQ ID NO: 4.

3. An isolated polypeptide of claim 1 consisting of the amino acid sequence set forth in SEQ ID NO: 6.

4. An isolated polypeptide of claim 1 consisting of the amino acid sequence set forth in SEQ ID NO: 7.

5. An isolated polypeptide of claim 1 consisting of the amino acid sequence set forth in SEQ ID NO: 8.

6. An isolated polypeptide of claim 1 consisting of the amino acid sequence set forth in SEQ ID NO: 9.

7. An isolated polypeptide of claim 1 consisting of the amino acid sequence set forth in SEQ ID NO: 10.

8. An isolated polypeptide of claim 1 consisting of the amino acid sequence set forth in SEQ ID NO: 11.

9. An isolated polypeptide of claim 1 consisting of the amino acid sequence set forth in SEQ ID NO: 12.

10. An isolated polypeptide of claim 1 consisting of the amino acid sequence set forth in SEQ ID NO: 13.

11. An isolated polypeptide of claim 1 consisting of the amino acid sequence set forth in SEQ ID NO: 14.

12. An isolated polypeptide of claim 1 consisting of the amino acid sequence set forth in SEQ ID NO: 15.

13. An isolated polypeptide of claim 1 consisting of the amino acid sequence set forth in SEQ ID NO: 16.

14. An isolated polypeptide of claim 1 consisting of the amino acid sequence set forth in SEQ ID NO: 17.

15. An isolated polypeptide of claim 1 consisting of the amino acid sequence set forth in SEQ ID NO: 18.

16. An isolated polypeptide of claim 1 consisting of the amino acid sequence set forth in SEQ ID NO: 19.

17. An isolated polypeptide of claim 1 consisting of the amino acid sequence set forth in SEQ ID NO: 20.

18. An isolated nucleic acid molecule that is at least 90% identical to a nucleic acid molecule consisting of the nucleic acid sequence set forth in SEQ ID NO: 2.

19. An isolated nucleic acid molecule of claim 18 that is at least 95% identical to a nucleic acid molecule consisting of the nucleic acid sequence set forth in SEQ ID NO: 2.

20. An isolated nucleic acid molecule of claim 18 that is at least 99% identical to a nucleic acid molecule consisting of the nucleic acid sequence set forth in SEQ ID NO: 2.

21. An isolated nucleic acid molecule of claim 18, wherein said isolated nucleic acid molecule consists of the amino acid sequence set forth in SEQ ID NO: 2.

22. An isolated polypeptide that is at least 90% identical to a polypeptide consisting of the amino acid sequence set forth in SEQ ID NO: 3.

23. An isolated polypeptide of claim 22, wherein said isolated polypeptide is at least 95% identical to the amino acid sequence set forth in SEQ ID NO: 3.

24. An isolated polypeptide of claim 22, wherein said isolated polypeptide is at least 99% identical to the amino acid sequence set forth in SEQ ID NO: 3.

25. An isolated polypeptide of claim 18, wherein said polypeptide consists of the amino acid sequence set forth in SEQ ID NO: 3.

26. A vector comprising a nucleic acid molecule of claim 18.

27. A vector of claim 26, wherein the nucleic acid molecule consists of the nucleic acid sequence set forth in SEQ ID NO: 2.

28. A plant cell comprising a vector of claim 26.

29. A plant cell comprising a vector of claim 27.

30. A method of enhancing meristem growth in a plant, the method comprising the steps of:
(a) introducing into a plant an expression vector comprising a nucleic acid molecule that encodes a RALF polypeptide; and
(b) expressing the RALF polypeptide within the plant.

31. A method of inhibiting meristem growth in a plant, the method comprising the steps of:
(a) introducing into a plant an expression vector that comprises a nucleic acid sequence that is transcriptionally expressed to yield a nucleic acid molecule that hybridizes under stringent conditions to a nucleic acid molecule consisting of a nucleic acid sequence selected from the group of sequences consisting of SEQ ID NO: 2, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35; and
(b) transcriptionally expressing the nucleic acid sequence in the plant.
